(19) United States
(12) Patent Application Publication
(10) Pub. No.: US 2021/0177732 A1
(43) Pub. Date: Jun. 17, 2021

(54) COMBINATION OF EXTRACTS OF QUINQUINA AND OF LEONTOPODIUM ALPINUM AND OF THE MANGANESE SALT OF L-PYRROLIDONE CARBOXYLIC ACID IN THE TREATMENT OF ALOPECIA

(71) Applicant: PIERRE FABRE DERMO-COSMETIQUE, Boulogne-Billancourt (FR)

(72) Inventors: Sylvie DAUNES-MARION, TOULOUSE (FR); Marguerite LEVEQUE, TOULOUSE (FR); Stéphane POIGNY, SAUBENS (FR); Anne MANDEAU, TOULOUSE (FR)

(73) Assignee: PIERRE FABRE DERMO-COSMETIQUE, Boulogne-Billancourt (FR)

(21) Appl. No.: 17/052,694

(22) PCT Filed: May 3, 2019
(86) PCT No.: PCT/EP2019/061445

§ 371 Date: Nov. 3, 2020

(30) Foreign Application Priority Data
May 4, 2018 (FR) 1853867

(51) Int. Cl. A61K 8/9789; A61Q 7/00; A61K 8/49

(52) U.S. Cl. CPC A61K 8/9789; A61K 8/4913; A61Q 7/00

(57) ABSTRACT

The present invention relates to a new combination comprising an extract of quinquina, an extract of Leontopodium alpinum and the manganese salt of L-pyrrolidonecarboxylic acid, and also to the use of said combination in the hair field, more particularly in the treatment or prevention of alopecia.

### TECHNICAL FIELD

The present invention relates to a novel combination of an extract of cinchona, the manganese salt of L-pyrrolidone carboxylic acid and an extract of Leontopodium alpinum, and to the use of said combination in the field of hair science, more particularly in the treatment or prevention of alopecia.

### PRIOR ART

Hair care, not only for cosmetic purposes but also to prevent hair loss and to regenerate hair, has always attracted researchers' minds. A number of theories have attempted to clarify the etiology of hair loss in cases of baldness, alopecia, pelade, etc., placing blame on seborrhea, an increase in the tension of the tissue on the cranial sphere, reduced blood irrigation, or certain endocrine or nerve conditions.

The hair follicle is a mini-organ anchored in the skin to the hypodermis, whose principal function is the production of a hair shaft. Their distribution is established during in utero growth and their number is genetically determined. The hair follicle is a dynamic structure that produces hair during cycles of growth and tissue remodeling. This cycle is divided into 3 phases:
A growth phase (anagen), the dermal papilla cells (fibroblasts) send a signal to the bulb stem cells which allows them to proliferate. These cells will transform and envelop the dermal papilla to form the sulfur matrix of the hair. They divide and differentiate into keratinocytes, the cells responsible for the structure of the hair. In order for the hair to be well-structured, the keratinocytes need sulfur-containing proteins, vitamin B6 and various minerals such as zinc and magnesium. The length of this phase determines the length of the hair and depends on the proliferation and differentiation of the matrix cells at the base of the follicle.
A regression phase (catagen), the matrix dies and consequently the dermal papilla loses contact with this matrix. Exchange between the cells stops. The follicle and the dermal papilla rise toward the epidermis.
A rest phase (telogen), the cells of the dermal papilla and of the bulb are intact and inactive. The hair falls out. For a new hair to develop, the cycle must be reinitiated.

The development and the growth of the hair follicle are influenced by compounds expressed by the dermal papilla, proteins such as Wnt and growth factors such as keratinocyte growth factor (KGF) or epithelial growth factor (EGF).

Hair is therefore continually being renewed, and of the 100 000 to 150 000 hairs that make up a head of hair, the majority are in the growth phase. There is a normal and physiological loss of about 60 to 100 hairs per day for healthy hair. Beyond that, hair loss is said to be pathological whether it is occasional or permanent.

The term alopecia refers to the partial or general loss of hair. Many factors can be involved in alopecia, such as, for example, genetic factors, age, sex, diseases, stress, hormonal problems, side effects of medication, scars. Several forms of alopecia can be distinguished:
Hereditary androgenetic alopecia, which is the most common. Early hair loss occurs in genetically predisposed individuals and affects men in particular. It is manifested by a decrease in hair volume, or even baldness, and affects 50% of men over 50 years of age;
Postmenopausal alopecia, which is the most common cause of baldness in women. In women, hair loss is more diffuse and extensive than in men. Female diffuse alopecia is a disorder that often begins at menopause and affects about 40% of women over 70. The term diffuse indicates that, unlike in men, hair loss affects the entire scalp in a uniform manner;
Acute or reactive alopecia, which can be linked to chemotherapy treatment, stress, childbirth, significant nutritional deficiencies, iron deficiency, hormonal disorders, is a simultaneous and diffuse loss of a large quantity of hair;
Scarring alopecia, which can be caused by skin problems (tumors, burns, pelade), acute radiation, lupus erythematosus or parasites (ringworm, lichen);
Alopecia areata, which seems to be of autoimmune origin and is characterized by relatively large patches in one or more places;
Congenital alopecia, which is rare and corresponds to a lack of roots or to hair anomalies (mutations).

Alopecia is essentially linked to a disruption in hair renewal which leads first to an acceleration in the frequency of cycles at the expense of hair quality and then of hair quantity. The most common phenomenon is a reduction in the growth cycle (anagen phase) due to a halt in cell proliferation. This results in a premature induction of the catagen phase, a greater number of hair follicles in the telogen phase, and consequently to greater hair loss. To fight alopecia, it is thus necessary to restart the hair cycle, for example, by activating the anagen phase.

To date, various products have been proposed to fight alopecia. Most combine several active principles likely to bring a beneficial action on the biological parameters involved in hair loss. Among the most commonly encountered active principles, we can cite by way of examples: vitamins such as vitamins A, E, B5, B6, C, H and PP; trace elements such as zinc, copper, magnesium, silicon, etc.; protein derivatives such as peptides, sulfur-containing amino acids (such as methionine, cystine, cysteine or derivatives); essential oils or extracts of plant origin of lipophilic or hydrophilic nature whose list is not exhaustive; antifungal agents such as piroctone olamine, undecyclinic derivatives, ciclopirox olamine, etc.; molecules of chemical synthesis known for their specific action on androgen receptors or on the activity of 5-α reductases. Minoxidil or 2,4-diamino-6-piperidinopyrimidine-3-oxide is today a reference in the treatment of androgenic alopecia. Despite the many theories evoked on its mechanism of action, it is not clearly elucidated. Moreover, its efficacy remains limited, even though a stabilization of hair loss has been observed in many clinical cases, due to the resumption of the alopecia process as soon as the treatment is stopped. Its restrictive daily use is likely to be the cause of undesirable side effects, such as localized skin reactions or systemic effects, noted in patients using it over the long term.

There is therefore a need for novel compounds or compositions useful for fighting alopecia.

### SUMMARY OF THE INVENTION

Surprisingly, the inventors discovered that the combination of extracts of cinchona and of Leontopodium alpinum and of the manganese salt of L-pyrrolidone carboxylic acid induced a synergy of action on the Wnt-bCat pathway, detailed in Example 1.

The invention therefore relates to a combination comprising an extract of cinchona, an extract of Leontopodium alpinum and of the manganese salt of L-pyrrolidone carboxylic acid.

### DEFINITIONS

The terms “Leontopodium alpinum” and “edelweiss” are equivalent and are used interchangeably.

“Extract of Leontopodium alpinum” means the product of extraction of all or part of the edelweiss plant.

“Extract of cinchona” means the product of extraction of one or more cinchona species, preferably obtained from cinchona bark.

“Product of extraction” means the product obtained after extraction of all or part of the plant to be extracted with a solvent, called extraction solvent, (i.e. a liquid solution in the extraction solvent) possibly in concentrated or dry form after partial or total evaporation of the extraction solvent.

In the context of the present invention, “polar to medium polar solvent” means a solvent having a dipole moment greater than or equal to 1.0 D. In particular, it may be a solvent selected from the group consisting of water, C1 to C5 alcohols (e.g. ethanol), C3 to C5 glycols (e.g. propylene glycol, butylene glycol, pentylene glycol), glycerol, acetone, C1 to C5 alkyl esters (e.g. ethyl acetate, isopropyl acetate), C1 to C5 halogenated hydrocarbons (e.g. chloroform, dichloromethane), and mixtures thereof.

In the context of the present invention, “hydroalcoholic extract” means an extract obtained with the aid of an extraction solvent consisting of a C1 to C5 alcohol/water mixture, such as an ethanol/water mixture.

In the context of the present invention, “dry extract” means an extract free of extraction solvent or carrier or containing only insignificant trace amounts thereof. Such a dry extract thus contains only material derived from the plant which has been extracted.

“Head and/or body hair” means head hair, body hair, eyebrows, eyelashes and/or coat, preferentially head hair.

“Alopecia” means the total or partial loss of head and/or body hair, for example due to reduced hair growth and/or to accelerated head and/or body hair loss. This term includes but is not limited to androgenetic alopecia, postmenopausal alopecia, reactive alopecia, scarring alopecia, alopecia areata, congenital alopecia. The consequences of alopecia are a temporary or permanent and partial or total absence of head and/or body hair.

The term “treat” alopecia means to stop alopecia, to reduce alopecia and/or to mitigate alopecia. Thus, “treating” alopecia includes limiting head and/or body hair loss, promoting head and/or body hair growth, increasing hair follicle density and/or regulating the phases of the hair follicle cycle.

The term “prevent” alopecia means to decrease the risk of developing alopecia, or to slow the progression of alopecia in a mammal, preferentially man, who is likely to develop alopecia.

The term “limit” means to slow down, to reduce, to diminish and/or to stop.

The term “promote” means to increase, enhance, favor, amplify and/or accelerate.

In the present invention, the term “cosmetically or dermatologically acceptable” means that which is useful in the preparation of a cosmetic or dermatological composition, which is generally safe, non-toxic and neither biologically nor otherwise undesirable, and which is acceptable for cosmetic or dermatological use, in particular by topical application and/or by oral administration.

### DETAILED DESCRIPTION

According to a first aspect, the invention relates to an association comprising an extract of cinchona, an extract of Leontopodium alpinum and the manganese salt of L-pyrrolidone carboxylic acid.

The cinchonas have long been known for their medicinal properties. It is the case in particular of the red cinchona (Cinchona succirubra), which is a small tree that can reach 10 m in height with a trunk of 20 cm in diameter, it keeps a green foliage all year round. It belongs to the Rubiaceae family, native to equatorial regions and more particularly to South America and whose bark is rich in quinine. Once harvested, the bark tends to become reddish brown on its inner side. Currently, cultures exist in Southeast Asia, South America and Africa. The bark is imported from Indonesia, India, Sri Lanka and partly from South America and Africa. The bark is obtained by debarking the roots, trunk and branches, the bark regenerates partially, it is not necessary to cut the tree.

To obtain the bark, the first step is to identify the cinchona according to need. This identification is done thanks to the shape of the leaves. The selection of the tree is carried out by taking into account the age and therefore the size of the branches. Only branches between 6 and 8 years old are selected. Moreover, the bark must be sufficiently thick. Thus, only bark that has never been harvested or that has regenerated is selected. After harvest, a cinchona bark regenerates in 2-3 years depending on the weather conditions. The bark is harvested by hand using a machete. It is done in dry weather from July to November. In order to preserve the plant, only the bark is now harvested without cutting the trunk. The harvested bark is packed in bags and transported to the drying location. Drying takes place about 20 days after the harvest. It consists of natural drying in the sun and lasts about 15 days. The barks are then cleaned to remove traces of moss. They are then selected according to their size. A screening is carried out to remove dust.

The extract of cinchona of the present invention is an extract of one or more species of cinchona, and preferably of the bark, selected from the forty or so species of cinchona known to date, between which hybridizations are numerous. Preferably, the cinchona according to the invention is selected from: the gray cinchonas (Chinchona officinalis), which are aromatic, low in tannin and in alkaloids; the yellow cinchonas (Cinchona calisaya) which are the highest in total alkaloids and quinine; the red cinchonas (Chinchona succirubra) which are high in tannin, and higher than the gray cinchonas in alkaloids; the cinchonas with quinine; and combinations thereof. Preferably, the cinchona according to the invention is selected from the red cinchonas.

Extracts of cinchona bark are known for certain therapeutic properties. For example, cinchona barks are astringent due to their tannin, bitter tonic, febrifuge and anti-malarial due to their alkaloids, particularly quinine. The antipyretic properties of the bark are mainly due to the quinine, and to a lesser extent to the alkaloids quinidine, cinchonine, cinchonidine. Cinchona is tonic thanks to its quinotannic acid which is partly combined with alkaloids. The tonic action is also due to quinovine.

In the present invention, the extract of cinchona is used as active principle and not as tonic.

In a preferred way, the extract of cinchona according to the invention is an ethanolic extract of cinchona bark, especially red cinchona.

The extract of cinchona according to the invention advantageously contains 1 to 10% by weight of quinine in relation to the weight of the dry extract. Furthermore, the extract of cinchona according to the invention advantageously contains between 4 and 20% by weight of total alkaloids (including quinine) in relation to the weight of the dry extract. The extract of cinchona according to the invention may also contain between 5 and 10% by weight of proanthocyans (polyphenols) expressed as procyanidin B2, in relation to the weight of the dry extract.

For example, the extract according to the present invention may be obtained by extraction with ethanol of dried and ground cinchona bark, in particular red cinchona, the mixture then being filtered to recover the extract. During extraction, the plant/ethanol volume ratio is for example comprised between 1/5 and 1/10, in particular between 1/7 and 1/9. After filtration, the extract can be stabilized by the addition of citric acid (e.g. 0.1 to 1% (w/v) such as 0.4% (w/v)). The extract thus obtained can be a reddish-brown liquid containing 3 to 5% dry matter. The extract thus obtained advantageously contains 0.06 to 0.3% (w/v) quinine. The extract also contains between 0.2 and 0.6% (w/v) of total alkaloids (including quinine) and about 0.3% (w/v) of proanthocyans (polyphenols) expressed as procyanidin B2. The extract obtained can be used as is or as a concentrated extract or dry extract after partial or total evaporation of the extraction solvent (ethanol).

The edelweiss (Leontopodium alpinum also called Leontopodium nivale) is a plant species of the Asteraceae family. It is one of the most famous mountain plants, partly because of its rarity in its natural environment, it grows at an altitude of 1500 to 3000 meters, in areas that are relatively inhospitable (ravines, rocky, cold and very exposed to UV). This plant has adapted perfectly to these extreme conditions through a panoply of molecules of interest and thanks to protective hairs on its flower and leaves. Indeed, its leaves are felted of woolly white hairs, its flowers are also felted of woolly white hairs with a characteristic inflorescence in assembly of 5 to 6 small yellow flower heads surrounded by leaflets arranged in star. This plant is found in the Alps, but also in the Pyrenees, the Carpathians and the Balkans. The edelweiss is partially protected, but this plant is also cultivated. Recently, man has mastered the culture of cells from very small parts of plants as a fragment of leaf, root, stem. This tool opens the way to the production of biomass and of molecules of interest.

The plant is not toxic, although inedible, decoctions of flowers in milk are still regularly prepared. This plant is used in folk medicine against abdominal pain, angina, bronchitis and diarrhea or dysentery. The cosmetics industry is interested in its antioxidant properties. Extracts of dried roots of Leontopodium alpinum have anti-inflammatory properties. The vast majority of the properties of this plant is attributed to the presence of secondary polyphenolic metabolites. Edelweiss contains a wide variety of polyphenols belonging to the classes of phenylpropanoids (phenolic acid, glycosides, flavonoids, coumarins and lignans), terpenes (sesquiterpenes and diterpenic acids) and alkaloids (benzofuran and pyran derivatives). Analytical evaluation of aerial portions of edelweiss reveals glycosides and aglycones of flavonoids (luteolin, quercetin, and apigenin) as well as leontopodic, chlorogenic, and 3,5-dicaffeoylquinic acids.

In the context of the present invention, the extract of Leontopodium alpinum or extract of edelweiss may be obtained from all or part of the plant (edelweiss) selected from the aerial parts such as leaves, stems, flowers, seeds; the subterranean parts such as roots; and combinations thereof. Advantageously it is the aerial parts.

The edelweiss plant or plant part can be fresh or dry, whole, cut or ground, and then subjected to an extraction step.

A process for preparing an extract according to the invention advantageously comprises a step of extraction of all or part of the Leontopodium alpinum plant by an extraction solvent advantageously selected from polar to medium polar solvents.

The extraction solvent will preferably be selected from the group consisting of water, C1 to C5 alcohols (e.g. ethanol), C3 to C5 glycols (e.g. propylene glycol, butylene glycol, pentylene glycol), glycerol, acetone, C1 to C5 alkyl esters (e.g. ethyl acetate, isopropyl acetate), halogenated, especially chlorinated, C1-C5 hydrocarbons (e.g. chloroform, dichloromethane), mixtures thereof.

In an embodiment of the invention, the extraction solvent is a C1 to C5 alkyl ester (e.g. ethyl acetate, isopropyl acetate), a C1 to C5 alcohol (e.g. ethanol), a C1 to C5 alcohol/water mixture (e.g. ethanol/water) or water. Advantageously, it will be an ethanol/water mixture, used for example in a volume ratio of 1/10 to 10/1.

According to a particular embodiment of the invention, the extraction is carried out under mixing or statically, at reflux, at room temperature, or at a temperature between room temperature and reflux. It can be assisted by ultrasound, by microwave, by flash expansion or by extrusion. The extraction can be carried out in a ratio weight of plants/volume of extraction solvent that can vary from 1/3 to 1/30, in particular for a time of 1 minute to 48 hours, for example from 10 minutes to 24 hours. The extraction can be repeated 2 to 3 times. The marc is then separated from the extract, in particular by centrifugation or filtration, and the solution can possibly be more or less concentrated notably up to a dry extract. A carrier can be added during the concentration step so as to obtain an extract containing 1 to 75% dry extract. The carrier can be maltodextrin, lactose, silica, glycerin, a glycol (e.g. 1,2-pentanediol, 1,3-butanediol, 1,3-propanediol), a vegetable oil, or any other carrier that is cosmetically acceptable and solubilizes the extract, preferentially of biosourced origin, or a mixture thereof. The extract can also be decolorized, for example on activated carbon, in order to eliminate all or part of the chlorophylls.

According to a preferred embodiment of the invention, the extract of Leontopodium alpinum is prepared as follows: the aerial parts of Leontopodium alpinum are dried under a flow of hot air and then ground. Then an extraction is carried out with an ethanol/water solution, used for example in a volume ratio of 1/10 to 10/1. The ethanol is then removed by vacuum distillation. The concentrate is formulated with glycerin as carrier and filtered.

The extracts of Leontopodium alpinum according to the invention can also be obtained using directed plant cell culture technology. By exposing the cultures to fine physical or nutritional variations for example, it is possible to modulate their metabolic processes and thus promote an increase in the synthesis of molecules of interest, such as leontopodic acid.

According to a particular embodiment of the invention, the extract of Leontopodium alpinum according to the invention is characterized by a content of 0.05 to 1% leontopodic acid, % by weight in relation to the weight of the dry extract, preferably at least 0.1% leontopodic acid, % by weight in relation to the weight of the dry extract.

Manganese is one of the trace elements essential to the good balance of the body. It is useful to the cells at very low doses, manganese contributes to the activity of many enzymes and in particular to that of superoxide dismutase. The body has several metal-dependent superoxide dismutases, but those activated by manganese are essential for skin protection. Indeed, located in the mitochondria, they are the body's first line of defense against superoxide radicals produced as a result of inflammation reactions or skin exposure to UV light. The induction of manganese-dependent superoxide dismutase can be regulated by the superoxide anions themselves but also by different cytokines such as IL-1α and TNFα. The dismutation reaction of the superoxide anions to hydrogen peroxide is spontaneous, but the manganese-dependent superoxide dismutase accelerates the rate and superoxide radicals are eliminated 10¹⁰ times faster.

Manganese is necessary for the proper functioning of the brain, manganese is effective in the treatment of many nerve disorders. Essential for metabolism and energy production (co-factor of the pyruvate kinase found in the Krebs cycle, of arginase, the enzyme that transforms arginine into urea), it also has a major role in the manufacture of proteins and nucleic acids.

L-Pyrrolidone carboxylic acid is considered as a moisturizing agent when it is associated with different cations (sodium, potassium, calcium, etc.), it enters at 12% in the composition of the natural skin moisturizing factor, the aqueous part of the hydrolipidic film of the surface of the epidermis. L-Pyrrolidone carboxylic acid occupies a central place in the biochemistry of the body and the skin, it is the link between energy metabolism, protein pool and skin hydration. From a biological point of view, L-pyrrolidone carboxylic acid is considered to be a natural transporter that carries the cations associated with it to the heart of the cells.

Thus, the manganese salt of L-pyrrolidone carboxylic acid constitutes a physiological contribution in bioavailable manganese in order to stimulate the activation of the manganese-dependent superoxide dismutase and to strengthen natural anti-free radical defenses of the skin with a dual preventive and curative effect. The manganese salt of L-pyrrolidone carboxylic acid has an efficacy that enables it to stimulate, in the absence of insult, the basal concentration of the skin in manganese-dependent superoxide dismutase and thus strengthens its level of anti-radical defense. Furthermore, it makes it possible to effectively fight free radicals by increasing the response of the skin exposed to UV radiation.

In a particular embodiment, the invention relates to a combination comprising an extract of red cinchona, in particular an extract of red cinchona bark, an extract of Leontopodium alpinum and the manganese salt of L-pyrrolidone carboxylic acid.

According to another particular embodiment, the invention relates to a combination comprising an extract of red cinchona, in particular an extract of red cinchona bark, a hydroalcoholic extract of Leontopodium alpinum and the manganese salt of L-pyrrolidone carboxylic acid.

According to a second aspect, the present invention also relates to a cosmetic or dermatological composition comprising a combination according to the invention according to an embodiment described above and at least one cosmetically or dermatologically acceptable excipient, more particularly adapted for topical application and/or for oral administration, preferably for topical application.

In a particular embodiment, the combination according to the invention is the sole anti-alopecia active principle of the composition.

In another particular embodiment, the cosmetic or dermatological compositions according to the invention comprise at least one other anti-alopecia active principle such as finasteride or minoxidil.

The invention preferably relates to cosmetic or dermatological compositions according to the invention in a form suitable for topical application.

The cosmetic or dermatological compositions according to the invention may thus be in the forms which are usually known for topical administration, i.e. in particular lotions, shampoos, balms, foams, gels, dispersions, emulsions, sprays, serums, masks or creams, with excipients allowing in particular penetration in order to improve the properties and accessibility of the active principles.

Advantageously, the compositions according to the invention may be in the forms that are usually known for topical administration to the hair and scalp, i.e. in particular a shampoo, a conditioner, a hair cream, a hair lotion, a mask or a leave-in spray.

A distinction is thus made between formulated products that can be rinsed and formulated products that do not require rinsing.

These compositions generally contain, in addition to the compounds and extracts of the combination according to the present invention, a physiologically acceptable medium, generally based on water or solvent, for example alcohols, ethers or glycols. They may also contain surfactants, complexing agents, preservatives, stabilizers, emulsifiers, thickeners, gelling agents, humectants, emollients, trace elements, essential oils, fragrances, dyes, moisturizing agents or thermal waters, etc.

Advantageously, the compositions according to the present invention will comprise 0.01% to 10% by weight, preferably 0.1 to 8% by weight, more preferably 0.5% to 6% by weight, more preferably 1% to 5% by weight of extract of cinchona, relative to the total weight of the composition. Preferably, the composition will comprise 3.0% by weight of extract of cinchona, relative to the total weight of the composition. Equally preferred, the composition will comprise 5.0% by weight of extract of cinchona, relative to the total weight of the composition. The extracts of cinchona used in the compositions according to the invention will advantageously comprise a dry extract content of 1% to 10%, preferably 1% to 5%, by weight relative to the total weight of the extract.

According to another embodiment, the compositions according to the present invention will comprise 0.0001% to 1%, preferably 0.001 to 0.8% by weight, preferably 0.005% to 0.6% by weight, even more preferably 0.01% to 0.5% of extract of cinchona, by weight of dry extract relative to the total weight of the composition. The extracts of cinchona used in the compositions according to the invention will advantageously comprise a dry extract content of 1% to 10%, preferably 1% to 5%, by weight relative to the total weight of the extract.

Advantageously, the compositions according to the present invention will comprise 0.01% to 5% by weight, preferably 0.05% to 2% by weight, preferably 0.1% to 1.0% by weight of extract of Leontopodium alpinism, relative to the total weight of the composition. Preferably, the composition will comprise 0.5% by weight of extract of Leontopodium alpinum, relative to the total weight of the composition. Equally preferably, the composition will comprise 1.0% by weight of extract of Leontopodium alpinum, relative to the total weight of the composition. The extracts of Leontopodium alpinum used in the compositions according to the invention will advantageously comprise a dry extract content of 1% to 20%, preferably 5% to 10%, by weight relative to the total weight of the extract.

According to another embodiment, the compositions according to the present invention will comprise 0.0001% to 1%, preferably 0.0005 to 0.5% by weight, preferably 0.005% to 0.6% by weight, more preferably 0.001% to 0.2%, even more preferably 0.005% to 0.1% of extract of Leontopodium alpinum, by weight of dry extract relative to the total weight of the composition. The extracts of Leontopodium alpinum used in the compositions according to the invention will advantageously comprise a dry extract content of 1% to 20%, preferably 5% to 10%, by weight relative to the total weight of the extract.

Advantageously, the compositions according to the present invention will comprise 0.01% to 1.5% by weight, preferably 0.01 to 1% by weight, preferably 0.05% to 0.5% by weight of manganese salt of L-pyrrolidone carboxylic acid, relative to the total weight of the composition. Preferably, the composition will comprise 0.1% by weight of manganese salt of L-pyrrolidone carboxylic acid, relative to the total weight of the composition. Equally preferred, the composition will comprise 0.5% by weight of manganese salt of L-pyrrolidone carboxylic acid, relative to the total weight of the composition.

According to a preferred embodiment, the compositions according to the present invention comprise:
0.01% to 10% by weight, preferably 0.1% to 8% by weight, preferably 0.5% to 6% by weight, more preferably 1 to 5% by weight of extract of cinchona, relative to the total weight of the composition, the extract of cinchona advantageously comprising a dry extract content of 1% to 10%, preferably 1% to 5%, by weight relative to the total weight of the extract;
0.01% to 5% by weight, preferably 0.05% to 2% by weight, preferably 0.1% to 1% by weight of extract of Leontopodium alpinum, relative to the total weight of the composition, the extract of Leontopodium alpinum advantageously comprising a dry extract content of 1% to 20%, preferably 5% to 10% by weight, based on the total weight of the extract;
0.01% to 1.5% by weight, preferably 0.01% to 1% by weight, more preferably 0.05% to 0.5% by weight, more preferably 0.05% to 0.2% by weight of manganese salt of L-pyrrolidone carboxylic acid, relative to the total weight of the composition.

According to another preferred embodiment, the compositions according to the present invention comprise:
0.0001% to 1%, preferably 0.001 to 0.8% by weight, preferably 0.005% to 0.6% by weight, more preferably 0.01% to 0.5% of extract of cinchona, by weight of dry extract relative to the total weight of the composition;
0.0001% to 1%, preferably 0.0005 to 0.5% by weight, preferably 0.005% to 0.6% by weight, more preferably 0.001% to 0.2%, even more preferably 0.005% to 0.1% of extract of Leontopodium alpinum, by weight of dry extract relative to the total weight of the composition;
0.01% to 1.5% by weight, preferably 0.01% to 1% by weight, preferably 0.05% to 0.5% by weight, more preferably 0.05% to 0.2% by weight of manganese salt of L-pyrrolidone carboxylic acid, relative to the total weight of the composition.

In particular, when the amounts of extract of cinchona, extract of Leontopodium alpinum and manganese salt of L-pyrrolidone carboxylic acid in the ternary combination according to the invention are expressed in parts, this combination comprises between 10 and 100 parts of extract of cinchona, between 1 and 10 parts of extract of Leontopodium alpinum and between 0.5 and 5 parts of manganese salt of L-pyrrolidone carboxylic acid. Preferably, the combination according to the invention comprises, in parts, 30 or 50 parts of extract of cinchona, 5 parts of extract of Leontopodium alpinum and 1 part of manganese salt of L-pyrrolidone carboxylic acid.

Preferably, the composition according to the invention has a light texture further allowing an optimal penetration without leaving the head and/or body hair oily. From the first applications, the hair will regain strength and vitality.

The compositions according to the invention can be manufactured according to processes well known to the person skilled in the art.

According to a third aspect, the invention relates to a combination according to the invention according to an embodiment described above or a cosmetic or dermatological composition according to the invention according to an embodiment described above, for use in the prevention and/or treatment of alopecia.

The invention also relates to the use of a combination according to the invention according to an embodiment described above or a cosmetic or dermatological composition according to the invention according to an embodiment described above, for the manufacture of a dermatological composition intended for the prevention and/or treatment of alopecia.

The invention also relates to a method for the prevention and/or treatment of alopecia comprising the administration to an individual in need thereof of an effective amount of a combination according to the invention according to an embodiment described above or of a cosmetic or dermatological composition according to the invention according to an embodiment described above.

The alopecia can be selected from the group consisting of androgenetic alopecia, postmenopausal alopecia, reactive alopecia and alopecia areata. More particularly, the alopecia is selected from the group consisting of androgenetic alopecia and reactive alopecia. Preferably, the alopecia is androgenetic alopecia.

In the context of the prevention and/or treatment of alopecia, the combination according to the invention or the cosmetic or dermatological composition according to the invention will be advantageously administered topically and/or orally.

The combination according to the invention or the cosmetic or dermatological composition according to the invention may be used in combination with a treatment for alopecia, such as finasteride or minoxidil, and/or in combination with compounds useful for a good hair structure, such as for example sulfur-containing proteins, vitamin B6 and various minerals such as zinc and/or magnesium.

The combination according to the invention or the cosmetic or dermatological composition according to the invention may be used in an individual who has undergone or is about to undergo a micrograft.

According to a fourth aspect, the invention relates to a cosmetic use of the combination according to the invention according to an embodiment described above or of the cosmetic or dermatological composition according to the invention according to an embodiment described above, to limit head and/or body hair loss and/or to promote hair growth and/or to increase hair follicle density and/or to obtain hair with greater coverage and/or to promote follicular regeneration.

The invention also relates to a cosmetic method for limiting head and/or body hair loss and/or for promoting hair growth and/or for increasing hair follicle density and/or for obtaining hair with greater coverage and/or for promoting follicular regeneration comprising the administration to an individual in need thereof of an effective amount of a combination according to the invention according to an embodiment described above or of a cosmetic or dermatological composition according to the invention according to an embodiment described above.

The combination according to the invention or the cosmetic or dermatological composition according to the invention will be advantageously administered topically and/or orally.

The combination according to the invention or the cosmetic or dermatological composition according to the invention may be used in combination with compounds useful for limiting head and/or body hair loss and/or for promoting hair growth and/or for increasing density and/or for obtaining hair with greater coverage and/or for promoting follicular regeneration and/or in combination with compounds useful for a good hair structure, such as for example sulfur-containing proteins, vitamin B6 and various minerals such as zinc and/or magnesium.

The combination according to the invention or the cosmetic or dermatological composition according to the invention thus makes it possible to stop hair loss, to prolong its cycle, so that the existing hair is preserved in quantity and in quality.

According to a fifth aspect, the present invention also relates to a cosmetic process for limiting head and/or body hair loss and/or promoting hair growth and/or increasing hair follicle density and/or obtaining hair with greater coverage and/or promoting follicular regeneration comprising a step of administering the combination according to the invention according to an embodiment described above or the cosmetic composition according to the invention according to an embodiment described above to an individual.

According to a particular embodiment, it is a cosmetic hair care process intended to improve the aesthetics of the hair by promoting hair growth and/or to obtain hair with greater coverage characterized in that it consists in applying to the hair and the scalp an effective amount of a combination according to the invention according to an embodiment described above or of a composition according to the invention according to an embodiment described above, leaving the latter in contact with the hair and the scalp, and optionally rinsing the hair and the scalp to eliminate said combination or composition.

The following examples illustrate the invention without limiting its scope.

### EXAMPLE 1

### Pharmacological Test of an Extract of cinchona, Manganese Salt of L-pyrrolidone Carboxylic Acid and an Extract of Leontopodium alpinum and their Combination

The aim of this study is to evaluate the effects of the combination of an extract of cinchona, manganese salt of L-pyrrolidone carboxylic acid and an extract of Leontopodium alpinum on the activation of the Wnt/β-catenin pathway on dermal papilla cells derived from human hair follicles.

The development and growth of the hair follicle is influenced by compounds expressed by the dermal papilla: proteins such as Wnt and growth factors such as keratinocytes (KGF). They are involved in intercellular communication pathways and are known to act on follicular keratinocytes.

During the transition from the rest phase to the growth phase, the stem cells in the bulge are activated by the Wnt signal which regulates the expression of their genes. An increase of intracellular β-catenin is detected in the base of the bulge. Hair regeneration begins.

Wnt is a family of glycoproteins whose name corresponds to the merging of Wg (wingless) and Int (integration site). The Wnt protein signaling pathway via β-catenin is called canonical, i.e. the preferred pathway. The Wnt signal activates hair regeneration and participates in its growth. Wnt binds to extracellular receptors allowing the stabilization of intracellular β-catenin, thus preventing its degradation by the proteasome. β-Catenin can then penetrate the nucleus and play a role as a co-activator of transcription factors and stimulate the expression of specific genes involved in hair growth such as the gene encoding KGFs.

Hair development depends on a signaling loop between keratinocytes and dermal papilla cells. The expression of Wnt in keratinocytes induces the increase of β-catenin in dermal papilla cells regulating signaling pathways including growth factors that guide hair morphogenesis. KGF is an important endogenous paracrine mediator in the development, differentiation and growth of the hair follicle. β-Catenin and KGF are strongly present in the anagen phase and then disappear. A loss of β-catenin expression stops the Wnt signal and induces the transition to the catagen phase.

### Experimental Protocol

The studies are carried out on human cells derived from the dermal papillae of the follicles of three donors. The cells are inoculated in 96-well plates and cultured for 24 h with the necessary supplements. The cells are incubated with specific reagents in order to be transfected with a lentivirus (expressing the luciferase gene under the control of the Wnt/β-catenin promoter (TCF/LEF transcriptional response element). The transfected cells are incubated for 24 hours with the products to be tested, i.e. either with an extract of cinchona (10 μg/ml) diluted in DMSO, or with the manganese salt of L-pyrrolidone carboxylic acid (500 μM) diluted in water, or with an extract of Leontopodium alpinum (30 μg/ml) also diluted in water, or with the combination of the three compounds at the same concentrations as above. The cinchona used in this study is harvested in Ecuador, in the forests of Echeandia. The extract of Leontopodium alpinum tested in this study comes from a variety called Leontopodium alpinum Helvetia. The extract tested corresponds to the commercial material Alpaflor® from the supplier DSM. The manganese salt of L-pyrrolidone carboxylic acid tested in this study corresponds to the commercial material Mangalidone® from the supplier Solabia. It is obtained by cyclisation of glutamic acid of plant origin. Its INCI name is manganese PCA and its CAS number is 29193-02-2. A positive control (Wnt3a protein at 10 nM) is also tested. Activation of the Wnt/β-catenin pathway is highlighted by the quantification of luminescence due to luciferase expression.

A statistical analysis is performed, an intergroup comparison is made by a repeated measures ANOVA followed by Dunnett's post-test on the raw data.

The following Table 1 shows the activation of the Wnt/β-catenin pathway after incubation of the individual compounds alone or in combination.

TABLE 1

| | | | Average of 3 donors | | |
|---|---|---|---|---|---|
| | | | (6 experiments) | | |
| Treatment | | | Average | | Stats vs. |
| Compounds tested | Conc | | RLU | SEM | control |
| Control | | | 410.2 | 38.5 | — |
| Wnt3a | 10 | nM | 2366.9 | 743.6 | p < 0.01 |
| Cinchona (Q) | 10 | μg/mL | 412.2 | 40.8 | p = NS |
| Mn-PCA (L) | 500 | μM | 735.2 | 135.5 | p < 0.05 |
| Edelweiss (E) | 30 | μg/mL | 722.1 | 104.8 | p < 0.05 |

| | | | | |
|---|---|---|---|---|
| Q + L + E | 10 + 500 + 30 | 1030.4 | 176.7 | p < 0.01 |

Conc: concentrations; SEM: standard error to the mean; stats: statistical analyses; Q: extract of cinchona; Mn-PCA: manganese salt of L-pyrrolidone carboxylic acid; E: extract of edelweiss, i.e. Leontopodium alpinum; Q + L + E: extract of cinchona + manganese salt of L-pyrrolidone carboxylic acid + extract of edelweiss.

Treatment of human cells derived from follicular dermal papillae with the positive control of the experimental conditions, Wnt3a at 10 nM, induces a strong statistically significant activation of the Wnt-β-catenin pathway (+451±93%, p<0.01 versus control). This expected result validates the test and the experimental conditions.

Treatment of human cells derived from follicular dermal papillae with the extract of cinchona at 10 μg/mL shows no significant modulation of the Wnt-β-catenin pathway.

Treatment of human cells derived from follicular dermal papillae with the manganese salt of L-pyrrolidone carboxylic acid at 500 μM induces significant activation of the Wnt-β-catenin pathway (+77±17%, p<0.05 versus control).

Similarly, under the same conditions, the extract of edelweiss tested at 30 μg/mL significantly activated the Wnt-β-catenin pathway (+75±8%, p<0.05 versus control).

Treatment of human cells derived from follicular dermal papillae with the combination of the extract of cinchona at 10 μg/mL, the manganese salt of L-pyrrolidone carboxylic acid at 500 μM and the extract of edelweiss at 30 μg/mL induced a strong statistically significant activation of the Wnt-β-catenin pathway (+148±19%, p<0.01 versus control). Table 2 below summarizes the statistical analysis performed between the different groups.

TABLE 2

| Treatment | | | Statistical analysis | |
|---|---|---|---|---|
| Compounds tested | Conc | | Versus control | Versus Q + L + E |
| Control | — | | — | p < 0.01 |
| Cinchona (Q) | 10 | μg/mL | p = NS | p < 0.01 |
| Mn-PCA (L) | 500 | μM | p < 0.05 | p < 0.01 |
| Edelweiss (E) | 30 | μg/mL | p < 0.05 | p < 0.05 |

| | | | |
|---|---|---|---|
| Q + L + E | 10 + 500 + 30 | p < 0.01 | — |

The inventors thus demonstrate that this combination (extract of cinchona, extract of Leontopodium alpinum and manganese salt of L-pyrrolidone carboxylic acid) induces a greater activation of the Wnt-β-catenin pathway than that of the three compounds taken in isolation. The inventors thus demonstrate a synergy of action by combining these three compounds.

1. A combination comprising an extract of cinchona, an extract of Leontopodium alpinum, and the manganese salt of L-pyrrolidone carboxylic acid.

2. The combination according to claim 1, wherein the extract of cinchona is an extract of red cinchona bark.

3. The combination according to claim 1, wherein the extract of Leontopodium alpinum is a hydroalcoholic extract.

4-6. (canceled)

7. A cosmetic or dermatological composition comprising as active principle the combination according to claim 1, with at least one cosmetically or dermatologically acceptable excipient.

8. The composition according to claim 7, wherein the extract of cinchona represents 0.01% to 10% by weight of the total weight of the composition.

9. The composition according to claim 7, wherein the manganese salt of L-pyrrolidone carboxylic acid represents 0.01% to 1.5% by weight of the total weight of the composition.

10. The composition according to claim 7, wherein the extract of Leontopodium alpinum represents 0.01% to 5% by weight of the total weight of the composition.

11. The composition according to claim 7, further comprising another anti-alopecia active principle.

12. The composition according to claim 7, being in a form suitable for topical application.

13-14. (canceled)

15. A cosmetic hair care process intended to improve the aesthetics of the hair by promoting hair growth and/or to obtain hair with greater coverage, consisting in applying to the hair and the scalp an effective amount of the combination according to claim 1 or a cosmetic composition comprising as active principle the combination according to claim 1, with at least one cosmetically acceptable excipient, leaving the latter in contact with the hair and scalp, and optionally rinsing the hair and scalp.

16. The composition according to claim 7, wherein the extract of cinchona represents 0.5% to 6% by weight of the total weight of the composition.

17. The composition according to claim 7, wherein the extract of cinchona represents 1% to 5% by weight of the total weight of the composition.

18. The composition according to claim 7, wherein the manganese salt of L-pyrrolidone carboxylic acid represents 0.01% to 1.0% by weight of the total weight of the composition.

19. The composition according to claim 7, wherein the manganese salt of L-pyrrolidone carboxylic acid represents 0.05% to 0.5% by weight of the total weight of the composition.

20. The composition according to claim 7, wherein the extract of Leontopodium alpinum represents 0.05% to 2% by weight of the total weight of the composition.

21. The composition according to claim 7, wherein the extract of Leontopodium alpinum represents 0.1% to 1% by weight of the total weight of the composition.

22. A method for the prevention and/or treatment of alopecia comprising the administration to an individual in need thereof of an effective amount of the combination according to claim 1.

23. The method according to claim 15, wherein the alopecia is selected from the group consisting of androgenetic alopecia, reactive alopecia, postmenopausal alopecia and alopecia areata.

24. A cosmetic method for limiting head and/or body hair loss and/or for promoting hair growth and/or for increasing hair follicle density and/or for obtaining hair with greater coverage and/or for promoting follicular regeneration comprising the administration to an individual in need thereof of an effective amount of the combination according to claim 1.

25. A method for the prevention and/or treatment of alopecia comprising the administration to an individual in need thereof of an effective amount of a dermatological composition comprising as active principle the combination according to claim 1, with at least one dermatologically acceptable excipient.

26. A cosmetic method for limiting head and/or body hair loss and/or for promoting hair growth and/or for increasing hair follicle density and/or for obtaining, hair with greater coverage and/or for promoting follicular regeneration comprising the administration to an individual in need thereof of an effective amount of a cosmetic composition comprising as active principle the combination according to claim 1, with at least one cosmetically acceptable excipient.
